(19) United States
(12) Patent Application Publication
(10) Pub. No.: US 2023/0173083 A1
(43) Pub. Date: Jun. 8, 2023

(54) ALBUMIN-BINDING PRODRUG FOR PREVENTING OR TREATING CANCER AND PHARMACEUTICAL COMPOSITION INCLUDING THE SAME

(71) Applicant: KOREA INSTITUTE OF SCIENCE AND TECHNOLOGY, Seoul (KR)

(72) Inventors: Kwangmeyung KIM, Seoul (KR); Hong Yeol YOON, Seoul (KR); In-Cheol SUN, Seoul (KR); Man Kyu SHIM, Seoul (KR); Hanhee CHO, Seoul (KR)

(73) Assignee: KOREA INSTITUTE OF SCIENCE AND TECHNOLOGY, Seoul (KR)

(21) Appl. No.: 17/735,371

(22) Filed: May 3, 2022

(30) Foreign Application Priority Data
Dec. 7, 2021 (KR) 10-2021-0173890

(51) Int. Cl. A61K 47/64; A61K 47/65; A61K 31/704; A61P 35/00

(52) U.S. Cl. CPC A61K 47/643; A61K 31/704; A61K 47/65; A61P 35/00

(57) ABSTRACT

Disclosed is a prodrug for preventing or treating cancer. The prodrug is mediated by albumin present in the blood to achieve high cancer selectivity for cancer and high stability. Also disclosed is a pharmaceutical composition for preventing or treating cancer including the prodrug. The anticancer prodrug and the pharmaceutical composition form a conjugate with albumin even without using a carrier or delivery vector when injected in vivo. Therefore, the anticancer prodrug and the pharmaceutical composition are effective in preventing or treating cancer, have increased in vivo half-lives, and can accumulate in cancer with increased efficiency to significantly alleviate the side effects of the anticancer drug.

### CROSS-REFERENCE TO RELATED APPLICATIONS

This application claims priority under 35 U.S.C. §119 to Korean Patent Application No. 10-2021-0173890 filed on Dec. 7, 2021 in the Korean Intellectual Property Office, the disclosure of which is incorporated herein by reference in its entirety.

### BACKGROUND OF THE INVENTION

### 1. Field of the Invention

The present invention relates to a prodrug for preventing or treating cancer that is mediated by albumin present in the blood to achieve high cancer selectivity for cancer and high stability, and a pharmaceutical composition for preventing or treating cancer including the prodrug as an active ingredient.

### 2. Description of the Related Art

Chemotherapy for cancer is still the first-line treatment option owing to its applicability to a wide range of cancers. Many anticancer drugs have been developed and applied clinically. However, conventional anticancer drugs are accompanied by serious side effects attributed to their toxicity to normal tissues as well as cancer tissues. The risk of side effects greatly restricts drug dosage.

As an approach to alleviating the toxicity of anticancer drugs, research has been conducted aimed at developing prodrugs of anticancer drugs with low cytotoxicity. Such prodrugs have been designed based on enzymatic differences between cancer tissues and normal tissues, hypoxic environments in solid cancers or cancer-specific antigens expressed on the surface of cancer cells.

However, since prodrugs are also non-specifically converted to cytotoxic substances after administration to patients, they still have a risk of side effects. Even though prodrugs have minimal side effects compared to anticancer drugs, they have a short in vivo half-life and low tumor accumulation owing to their small molecule structure, resulting in limited anticancer efficacy.

Albumin is a protein present in the blood and has been considered as a carrier to improve the pharmacokinetic (PK) profile of anticancer drugs. However, in practice, the delivery efficiency of albumin-anticancer drug conjugates to cancer tissues is as considerably low as less than 1-3% and there still exists a risk of systemic toxicity due to albumin-anticancer drug conjugates delivered to normal tissues along the bloodstream. Considering that albumin-anticancer drug conjugates may cause side effects in patients, there is an urgent need to develop a new therapy that can maintain the in vivo anticancer efficacy of albumin-anticancer drug conjugates for a long period of time with minimal toxicity to normal tissues.

### Prior Art Documents

### Patent Documents

Patent Document 1. Korean Patent Publication No. 10-2021-0086852

### SUMMARY OF THE INVENTION

The present invention has been made in an effort to solve the above-described problems and one object of the present invention is to provide an anticancer prodrug that targets cancer cells.

A further object of the present invention is to provide a pharmaceutical composition for preventing or treating cancer including the anticancer prodrug as an active ingredient.

One aspect of the present invention provides an anticancer prodrug including a peptide having the amino acid sequence set forth in SEQ ID NO: 1, a maleimide group conjugated to the N-terminus of the peptide, and an anticancer drug conjugated to the C-terminus of the peptide.

The anticancer prodrug may further include a linker between the N-terminus of the peptide and the maleimide group.

The linker may be represented by *—(CH₂)₂—C(═O)—NH—(CH₂CH₂O)ₙ—CH₂CH₂—C(═O)—* wherein n is an integer from 1 to 10 and each asterisk * represents a binding site.

The anticancer prodrug may have a molecular weight of 1 to 5 kDa.

The anticancer prodrug may covalently bind selectively and rapidly to endogenous albumin present in the blood in situ to form an albumin-prodrug conjugate when administered intravenously.

The albumin-prodrug conjugate may have a molecular weight of 65 to 70 kDa.

The albumin-prodrug conjugate may have an in vivo half-life of 2 to 5 hours.

The anticancer prodrug may be activated by cathepsin B overexpressed in cancer cells.

The anticancer drug may be selected from the group consisting of doxorubicin, cyclophosphamide, mechlorethamine, uramustine, melphalan, chlorambucil, ifosfamide, bendamustine, carmustine, lomustine, streptozocin, busulfan, dacarbazine, temozolomide, thiotepa, altretamine, duocarmycin, cisplatin, carboplatin, nedaplatin, oxaliplatin, satraplatin, triplatin tetranitrate, 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine, camptothecin, topotecan, irinotecan, etoposide, teniposide, mitoxantrone, paclitaxel, docetaxel, ixabepilone, vinblastine, vincristine, vindesine, vinorelbine, estramustine, maytansine, mertansine (DM1), DM4, dolastatin, auristatin E, auristatin F, monomethyl auristatin E, monomethyl auristatin F, derivatives thereof, and combinations thereof.

The anticancer prodrug may be represented by Formula 1:

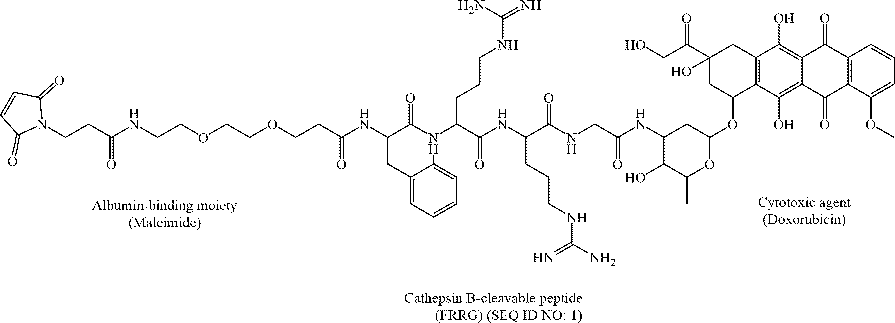

A further aspect of the present invention provides a pharmaceutical composition for preventing or treating cancer including the anticancer prodrug as an active ingredient.

The pharmaceutical composition may be administered by intravenous injection.

The cancer may be selected from the group consisting of gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, cervical cancer, bone cancer, non-small cell bone cancer, hematologic malignancy, skin cancer, head or neck cancer, uterine cancer, rectal cancer, perianal cancer, fallopian tube cancer, endometrial cancer, vaginal cancer, vulvar cancer, Hodgkin’s disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater cancer, peritoneal cancer, tongue cancer, small cell cancer, pediatric lymphoma, neuroblastoma, duodenal cancer, ureteral cancer, astrocytoma, meningioma, renal pelvis cancer, vulvar cancer, thymus cancer, central nervous system (CNS) tumor, primary central nervous system lymphoma, spinal cord tumor, brainstem glioma, pituitary adenoma, and combinations thereof.

The anticancer prodrug and the pharmaceutical composition according to the present invention form a conjugate with albumin even without using a carrier or delivery vector when injected in vivo. Therefore, the anticancer prodrug and the pharmaceutical composition according to the present invention are effective in preventing or treating cancer, have increased in vivo half-lives, and can be delivered to cancer with increased efficiency to significantly alleviate the side effects of the anticancer drug.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 schematically shows the working principle of a prodrug (Al-ProD). a: Chemical structure of the prodrug (Al-ProD), b: procedure for the formation of a conjugate by binding of the intravenously injected prodrug (Al-ProD) to in situ albumin in blood vessels, c: mechanism of action of the prodrug (Al-ProD) in malignant cancer cells, specifically an entire procedure where the prodrug is effectively accumulated in cancer cells and the anticancer drug is released by cathepsin B present in the cancer cells to exhibit anticancer efficacy, d: mechanism of action of the prodrug (Al-ProD) in normal cells after formation of a conjugate with albumin in vivo, showing that albumin-bound Al-proD exists in an stable inactive state because cathepsin B is hardly expressed in normal cells;

FIG. 2 schematically shows a synthetic scheme to prepare a prodrug (Al-ProD);

FIG. 3 shows the results of HPLC analysis for a prodrug (Al-ProD) synthesized in Example 1;

FIG. 4 shows the results of MALDI-TOF analysis for a prodrug (Al-ProD) synthesized in Example 1;

FIG. 5 shows the results of SDS-PAGE after incubation of a prodrug (Al-ProD) synthesized in Example 1 with albumin (HSA, MSA, BSA or blocked albumin) for 1 hour;

FIG. 6 shows the results of MALDI-TOF analysis after incubation of a prodrug (Al-ProD) synthesized in Example 1 with albumin (HSA, MSA, BSA or blocked albumin) for 1 hour;

FIG. 7 shows the results of RP-HPLC analysis to determine whether an albumin-prodrug conjugate was formed by incubation of a prodrug (Al-ProD) synthesized in Example 1 with HSA or thiol-blocked HSA for various times (0 min, 5 min, and 60 min);

FIG. 8 shows the results of HPLC analysis after an albumin-prodrug conjugate (Al-ProD+HSA) prepared in Example 2 was incubated with cathepsin B for various times (0 h, 3 h, 6 h, and 9 h);

FIG. 9 shows the results of HPLC analysis after an albumin-prodrug conjugate (Al-ProD+HSA) prepared in Example 2 was incubated with various enzymes (cathepsin E, cathepsin D, caspase-9, and caspase-3) for various times (0 h, 3 h, 6 h, and 9 h);

FIG. 10 shows the results of MALDI-TOF mass spectrometry after an albumin-prodrug conjugate (Al-ProD+HSA) prepared in Example 2 was incubated with cathepsin B for 9 h;

FIG. 11 shows confocal laser scanning microscopy (CLSM) images revealing the intracellular behaviors of doxorubicin (DOX) when MDA-MB-231, H9C2, and cathepsin B inhibitor-treated MDA-MB-231 were treated with an albumin-prodrug conjugate (Al-ProD+HSA) prepared in Example 2;

FIG. 12 shows the quantification analysis of DOX fluorescence in the nuclei or cytosol of MDA-MB-231, H9C2, and cathepsin B inhibitor-treated MDA-MB-231 after treatment with an albumin-prodrug conjugate (Al-ProD+HSA) prepared in Example 2;

FIG. 13 shows confocal laser scanning microscopy (CLSM) images revealing the intracellular behaviors of doxorubicin (DOX) when MDA-MB-231, H9C2, and cathepsin B inhibitor-treated MDA-MB-231 were treated with free DOX;

FIG. 14 shows the cytotoxicities of an albumin-prodrug conjugate (Al-ProD+HSA) prepared in Example 2 to H9C2 and MDA-MB231 cells;

FIG. 15 shows the cytotoxicities of free DOX to H9C2 and MDA-MB231 cells;

FIG. 16 shows the pharmacokinetics (PK) of a prodrug (Al-ProD) synthesized in Example 1 (Group 2) or free DOX (3 mg/kg, Group 1) after intravenous administration to cancer animal models;

FIG. 17 shows noninvasive near-infrared fluorescence (NIRF) images of groups intravenously administered a prodrug (Al-ProD) synthesized in Example 1 or free DOX (3 mg/kg);

FIG. 18 shows the quantification analysis of fluorescence from tumor tissues in NIRF images of groups intravenously administered a prodrug (Al-ProD) synthesized in Example 1 or free DOX (3 mg/kg);

FIG. 19 shows (right) noninvasive near-infrared fluorescence (NIRF) images of major organs (liver, lung, spleen, kidney, and heart) and cancer tissues excised from groups 12 hours after intravenous administration of a prodrug (Al-ProD) synthesized in Example 1 or free DOX (3 mg/kg) and (left) a graph showing the fluorescence in the images;

FIG. 20 shows confocal laser scanning microscopy (CLSM) images of DAPI-stained cancer tissues excised from groups intravenously administered a prodrug (Al-ProD) synthesized in Example 1 or free DOX (3 mg/kg);

FIG. 21 shows time-dependent changes in the cancer volume (V; mm³) of animal cancer models after treatment with a prodrug (Al-ProD) synthesized in Example 1, free DOX or physiological saline;

FIG. 22 shows time-dependent changes in the body weight of cancer animal models after treatment with a prodrug (Al-ProD) synthesized in Example 1, free DOX or physiological saline;

FIG. 23 shows time-dependent changes in the survival of cancer animal models after treatment with a prodrug (Al-ProD) synthesized in Example 1, free DOX or physiological saline;

FIG. 24 shows optical images of H&E- and TUNEL-stained sections of cancer tissues excised from cancer animal models sacrificed on day 20 after treatment with a prodrug synthesized in Example 1 (Al-ProD), free DOX or physiological saline; and

FIG. 25 shows optical images of H&E-stained sections of organ tissues excised from cancer animal models sacrificed on day 20 after treatment with a prodrug synthesized in Example 1 (Al-ProD), free DOX or physiological saline.

### DETAILED DESCRIPTION OF THE INVENTION

The present invention will now be described in detail.

As described above, conventional anticancer drugs have serious side effects such as systemic toxicity due to their very low selectivity for cancer cells. To solve this problem, various prodrugs and albumin-anticancer drug conjugates have been developed. However, they tend to degrade before reaching cancer cells, fail to show sufficient efficacy due to their unsuitable pharmacokinetics, and are still toxic to normal cells, limiting their practical use as therapeutic drugs.

As a solution to the above-described problems, the present invention intends to provide an albumin-binding anticancer prodrug with significantly low toxicity, an outstanding ability to target cancer cells, and extended in vivo residence time compared to existing anticancer drugs, and a pharmaceutical composition including the anticancer prodrug.

The present invention is directed to an anticancer prodrug including a peptide having the amino acid sequence set forth in SEQ ID NO: 1: Phe-Arg-Arg-Gly, a maleimide group conjugated to the N-terminus of the peptide, and an anticancer drug conjugated to the C-terminus of the peptide.

In the anticancer prodrug of the present invention, the anticancer drug is conjugated to the C-terminus of the peptide having the amino acid sequence set forth in SEQ ID NO: 1 and the maleimide group is conjugated to the N-terminus of the peptide, optionally via a linker.

As used herein, the term “peptide” refers to a linear molecule formed by amino acid residues bound to each other via peptide linkages. Representative amino acids and their abbreviations are as follows: alanine (Ala, A), isoleucine (Ile, I), leucine (Leu, L), methionine (Met, M), phenylalanine (Phe, F), proline (Pro, P), tryptophan (Trp, W), valine (Val, V), asparagine (Asn, N), cysteine (Cys, C), glutamine (Gln, Q), glycine (Gly, G), serine (Ser, S), threonine (Thr, T), tyrosine (Try, Y), aspartic acid (Asp, D), glutamic acid (Glu, E), arginine (Arg, R), histidine (His, H), and lysine (Lys, K).

An alteration in the amino acid sequence of the peptide in the anticancer prodrug may bring about a change in the overall structure of the prodrug, which may negatively affect the function of the prodrug. It is most preferable that the peptide used in the anticancer prodrug has the amino acid sequence set forth in SEQ ID NO: 1.

The peptide is cleaved by cathepsin B. More specifically, the G-anticancer drug is cleaved from the FRRG(SEQ ID NO: 1)-anticancer drug by cathepsin B overexpressed in cancer cells. Generally, binding of a macromolecule such as albumin with a prodrug may interfere with enzymatic activation of the prodrug due to steric hindrance or steric rejection. In contrast, the anticancer prodrug of the present invention covalently binds selectively and rapidly to albumin present in blood vessels in situ to form an albumin-prodrug conjugate when administered in vivo. The conjugate formation maximizes the pharmacokinetics (PK), anticancer efficacy, and stability of the anticancer prodrug against cancer cells. That is, the anticancer prodrug of the present invention is designed in comprehensive consideration of the conjugation relationship between the peptide and the anticancer drug, the binding relationship between the maleimide group and albumin, and their functions. Considering that conventional anticancer drugs lose their delivery efficiency when bound to albumin, the anticancer prodrug of the present invention is recognized to have a constitution and effects that cannot be achieved by conventional anticancer drugs or prodrugs.

The peptide and the maleimide group may be linked to each other via a linker. The linker is not particularly limited but is preferably represented by *—(CH₂)₂—C(═O)—NH—(CH₂CH₂O)ₙ—CH₂CH₂—C(═O)—* wherein n is an integer from 1 to 10 and each asterisk * represents a binding site.

The albumin may be bovine serum albumin (BSA), mouse serum albumin (MSA) or human serum albumin (HSA), preferably endogenous human serum albumin (HSA).

The anticancer prodrug of the present invention is stably present in vitro and forms a conjugate with albumin present in blood vessels when administered in vivo. The conjugate is stable with minimal side effects in vivo and is specifically activated against cancer cells despite its macromolecular structure. In addition, the conjugate exhibits greatly improved in vivo pharmacokinetics, achieving high therapeutic efficacy.

The anticancer prodrug is activated by an enzyme overexpressed in cancer cells and exhibits specific toxicity to the cancer. Accordingly, the anticancer prodrug is very stable without inducing cytotoxicity to normal tissues.

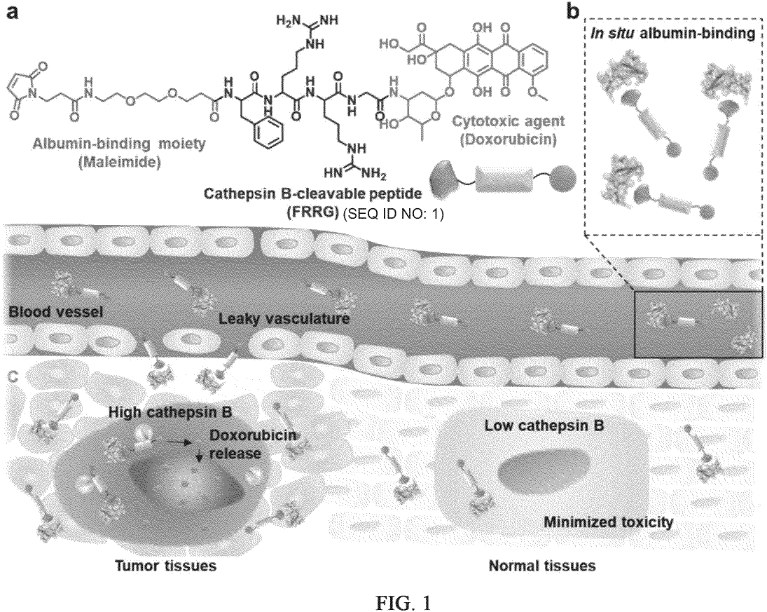

The mechanism of action of the anticancer prodrug according to the present invention is shown in FIG. 1. Referring to FIG. 1, the anticancer prodrug has a structure in which a peptide specifically degraded by cathepsin B is conjugated with an anticancer drug and a maleimide group. When administered in vivo, the anticancer prodrug binds to albumin to form a conjugate that is specifically activated in cancer cells. Specifically, when the peptide is degraded by cathepsin B, one of the degradation products is conjugated with the anticancer drug. The conjugated anticancer drug moves into the nuclei of cancer cells and exhibits high cytotoxicity to the cancer cells to effectively induce apoptosis in the cancer cells.

Although the anticancer prodrug of the present invention is a small molecule drug with a molecular weight of 1 to 5 kDa, it is stable and effectively accumulated in cancer cells when bound to albumin in vivo, as described above. In addition, the anticancer prodrug of the present invention can be retained in vivo for a long time, resulting in an at least 3-fold reduction in the frequency of administration compared to conventional anticancer drugs while avoiding continuous and repeated administration.

When even a very stable drug is administered in excess, its low toxicity is accumulated, eventually leading to increasing risks. This problem can be avoided by the use of the anticancer prodrug according to the present invention that has very high selectivity for cancer cells.

The anticancer prodrug of the present invention is synthesized in a simple manner under easily controlled conditions and does not require a separate process for formulation, such as encapsulation or attachment, facilitating its mass production and quality control (QC).

The cathepsin B is specifically overexpressed in cancer cells and its expression level is 20 to 30 times higher in cancer cells than in normal cells. As a result, when administered intravenously, the anticancer prodrug of the present invention covalently binds selectively and rapidly to albumin present in blood vessels in situ to form an albumin-prodrug conjugate, which can selectively accumulate in cancer tissues. In addition, the anticancer prodrug of the present invention is stable in normal cells where the expression of cathepsin B is low but exhibits high toxicity to cancer cells where the expression level of cathepsin B is high, thus being effective in preventing or treating cancer.

The albumin-prodrug conjugate may have a molecular weight of 65 to 70 kDa. The albumin-prodrug conjugate does not require a complicated synthetic process despite its increased molecular weight, has high accumulation in cancer tissues, and is effectively degraded by cathepsin B in cancer cells without steric hindrance, achieving superior anticancer efficacy compared to conventional anticancer drugs.

The albumin-prodrug conjugate has an in vivo half-life of 2 to 5 hours and is retained in vivo for 1 to 150 hours.

The anticancer prodrug of the present invention is not particularly dependent on the hydrophobicity of the anticancer drug because it can achieve excellent effects such as stability even without the need to form nanoparticles. The anticancer drug may be any of those used in conventional anticancer therapies but is preferably selected from the group consisting of doxorubicin, cyclophosphamide, mechlorethamine, uramustine, melphalan, chlorambucil, ifosfamide, bendamustine, carmustine, lomustine, streptozocin, busulfan, dacarbazine, temozolomide, thiotepa, altretamine, duocarmycin, cisplatin, carboplatin, nedaplatin, oxaliplatin, satraplatin, triplatin tetranitrate, 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine, camptothecin, topotecan, irinotecan, etoposide, teniposide, mitoxantrone, paclitaxel, docetaxel, ixabepilone, vinblastine, vincristine, vindesine, vinorelbine, estramustine, maytansine, mertansine (DM1), DM4, dolastatin, auristatin E, auristatin F, monomethyl auristatin E, monomethyl auristatin F, derivatives thereof, and combinations thereof. The anticancer drug is preferably doxorubicin.

The anticancer prodrug may be represented by Formula 1:

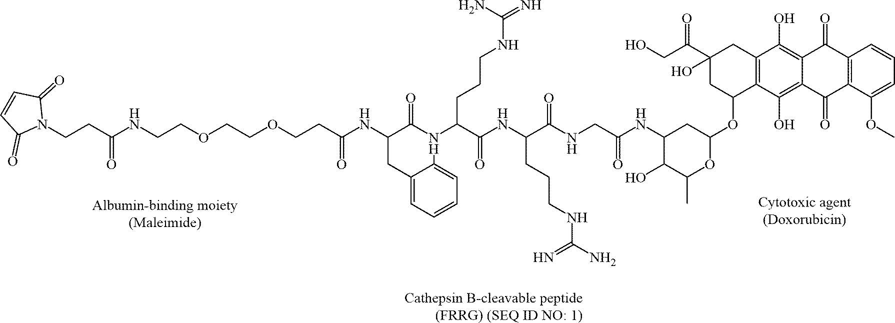

When injected in vivo, the anticancer prodrug binds to albumin present in blood vessels to form a conjugate, which exists very stably in vivo. When the conjugate is delivered into cancer cells, the peptide of the anticancer prodrug is degraded to albumin-FRR and a glycine (G)-conjugated anticancer drug by cathepsin B overexpressed in the cancer cells. The G-anticancer drug is delivered into the cell nuclei and converted into the anticancer drug in free form that induces apoptosis. Meanwhile, the peptide of the anticancer prodrug is not substantially degraded in normal cells where cathepsin B is present at a low concentration, and as a result, apoptosis is hardly induced. That is, the anticancer prodrug is maintained in a stable state in normal cells.

Since the anticancer prodrug of the present invention works in the cancer microenvironment, that is, by cathepsin B overexpressed in cancer cells, including resistant cancer, recurrent cancer, and metastatic cancer cells as well as general cancer cells. As used herein, the term “metastatic cancer” refers to a cancer caused by cancer cells that have spread from the primary organ to a distant organ and proliferated in the distant organ. The metastatic cancer is preferably invasion of cancer cells from the primary organ into a surrounding organ or metastasis of cancer cells to a different organ through blood or lymphatic vessels but is not particularly limited thereto.

As used herein, the term “recurrent cancer” refers to a cancer that has come back at the same site as the original cancer after a patient has been judged to be cured by initial treatment.

As used herein, the term “resistant cancer” refers to a cancer that exhibits extremely low sensitivity to cancer therapies such as radiotherapy or drugs for cancer treatment, particularly anticancer drugs, and whose symptoms are not ameliorated, alleviated, mitigated or treated by the therapy. The resistant cancer may be originally resistant to a specific therapy. Alternatively, the resistant cancer may not originally resistant to a specific therapy but no longer exhibit sensitivity to the same therapy due to genetic mutations in cancer cells caused by long-term treatment.

The cancer may be selected from the group consisting of gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, cervical cancer, bone cancer, non-small cell bone cancer, hematologic malignancy, skin cancer, head or neck cancer, uterine cancer, rectal cancer, perianal cancer, fallopian tube cancer, endometrial cancer, vaginal cancer, vulvar cancer, Hodgkin’s disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater cancer, peritoneal cancer, tongue cancer, small cell cancer, pediatric lymphoma, neuroblastoma, duodenal cancer, ureteral cancer, astrocytoma, meningioma, renal pelvis cancer, vulvar cancer, thymus cancer, central nervous system (CNS) tumor, primary central nervous system lymphoma, spinal cord tumor, brainstem glioma, pituitary adenoma, and combinations thereof. The anticancer prodrug of the present invention can exhibit significant anticancer effects on two or more carcinomas as well as one carcinoma.

The anticancer prodrug of the present invention binds to albumin in vivo to form a more stable conjugate. The conjugate formation enhances the stability of the drug and allows the drug to have high specific response to cancer. The conjugate extends the residence time of the anticancer prodrug by at least 3-fold due to its increased in vivo half-life.

A further aspect of the present invention is directed to a pharmaceutical composition for preventing or treating cancer including the anticancer prodrug as an active ingredient.

The composition of the present invention targets cancer cells, is selectively degraded by cathepsin B secreted from cancer cells or tissues to release the anticancer drug, and is minimally lost in the blood by normal cells and the reticuloendothelial system. The anticancer drug is activated in the nuclei of cancer cells targeted by the pharmaceutical composition of the present invention. This makes the pharmaceutical composition therapeutically effective and allows the pharmaceutical composition to effectively act against carcinomas that are resistant and refractory to conventional cancer therapies.

As used herein, the term “prevent”, “preventing” or “prevention” means all actions that inhibit or delay the cancer disease by administration of the pharmaceutical composition according to the present invention.

As used herein, the term “treat”, “treating” or “treatment” means all actions that improve or beneficially change symptoms of the cancer disease by administration of the pharmaceutical composition according to the present invention.

The pharmaceutical composition of the present invention may be administered in a pharmaceutically effective amount. The pharmaceutically effective amount is intended to include a “therapeutically effective amount” and a “prophylactically effective amount”. The term “therapeutically effective amount” means any amount of a drug or therapeutic agent that, when used alone or in combination with another therapeutic agent, decreases the severity of disease symptoms, increases the frequency and duration of disease symptom-free periods, or prevents impairment or disability due to the disease affliction. The term “prophylactically effective amount” means any amount of a drug that inhibits the development, metastasis or recurrence of cancer in an individual at risk of cancer development or an individual at risk of suffering from cancer metastasis or recurrence. The level of the effective amount may be determined by various factors, including the type of the individual, the severity of the disease, the age and sex of the individual, the activity of the drug, the sensitivity to the drug, the time and route of administration, the rate of excretion, the duration of treatment, and the type of concurrent drugs, and other factors well known in the medical field.

The dose of the pharmaceutical composition according to the present invention may vary depending on the patient’s age, sex, and body weight. Specifically, the pharmaceutical composition of the present invention may be administered in single or divided doses at preferred intervals, for example, at intervals of 3 days, depending on the progression of disease in an individual with cancer.

The pharmaceutical composition of the present invention may be administered to an individual via various routes, for example, orally or parenterally. Examples of suitable parenteral routes of administration include, but are not limited to, intravenous, intraperitoneal, intramuscular, intraarterial, buccal, intracardiac, intramedullary, intradural, transdermal, intraintestinal, subcutaneous, sublingual, and topical administration. Preferably, the pharmaceutical composition of the present invention is administered intravenously by injection.

The intravenous administration refers to intravenous injection. It is most preferable that the pharmaceutical composition of the present invention is administered intravenously for binding of the anticancer prodrug to albumin in blood vessels.

The content of the active ingredient in the composition of the present invention does not need to be particularly limited but is 1 µM or more, preferably 1 to 100 µM, more preferably 1 to 50 µM, most preferably 1 to 10 µM. When the anticancer prodrug is present in the amount defined above, the composition of the present invention has no toxicity in normal cells and induces toxicity in cancer cells, thus being effective in ameliorating, treating or preventing cancer.

The pharmaceutical composition of the present invention is administered in an amount of 0.01 to 10,000 mg/kg/day, preferably 0.1 to 200 mg/kg/day. The pharmaceutical composition of the present invention may be administered in single or divided doses per day.

The pharmaceutical composition of the present invention may be administered individually or in combination with other therapeutic agents. The pharmaceutical composition of the present invention may be administered sequentially or simultaneously with other therapeutic agents. The other therapeutic agents may be drugs such as compounds and proteins that promote cancer regression or further prevent tumor growth and are intended to include anticancer therapies such as radiotherapy other than medication.

The pharmaceutical composition of the present invention can be formulated with one or more pharmaceutically acceptable carriers in accordance with methods that can be easily carried out by those skilled in the art. The pharmaceutical composition can be provided in unit dosage forms or dispensed in multi-dose containers.

The pharmaceutically acceptable carriers are those that are commonly used for formulation. Examples of the pharmaceutically acceptable carriers include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oil. The pharmaceutical composition of the present invention may further include one or more additives selected from the group consisting of lubricating agents, wetting agents, sweetening agents, flavoring agents, emulsifying agents, suspending agents, and preservatives. Details of suitable pharmaceutically acceptable carriers and formulations can be found in Remington’s Pharmaceutical Sciences (19th ed., 1995).

Another aspect of the present invention provides a method for preventing or treating cancer, including administering the pharmaceutical composition to an individual with cancer.

The terms used in the method of the present invention have the same meanings as those defined in the pharmaceutical composition, unless otherwise specified.

As used herein, the term “individual” is meant to include any human or non-human animal. The term “non-human animal” is meant to include: vertebrates such as non-human primates; sheep; dogs; and rodents such as mice, rats, and guinea pigs. The individual is preferably a human. The ‘subject’ may be used interchangeably with ‘subject’ or ‘patient’ in the present invention.

The individual is preferably a human. The term “individual” is interchangeably used herein with the term “subject” or “patient”.

As a result of the cancer treatment, tumor growth may be inhibited by at least about 10%, at least about 20%, at least about 40%, at least about 60%, or at least about 80% in the treated individual compared to in an untreated individual.

Since the pharmaceutical composition used in the method of the present invention itself has high specificity for cancer cells, it may be administered via any general route as long as it can reach a target tissue. The pharmaceutical composition of the present invention may be administered intraperitoneally, intravenously, intramuscularly, subcutaneously, intradermally, orally, intranasally, intrapulmonarily or rectally according to the intended purpose. The pharmaceutical composition is preferably administered intravenously. However, there is no particular restriction on the route of administration of the pharmaceutical composition.

The present invention will be explained in more detail with reference to the following examples. However, these examples are not to be construed as limiting or restricting the scope and disclosure of the invention. It is to be understood that based on the teachings of the present invention including the following examples, those skilled in the art can readily practice other embodiments of the present invention whose experimental results are not explicitly presented. Such modifications and variations are intended to come within the scope of the appended claims.

The experimental results of the following examples, including comparative examples, are merely representative and the effects of the exemplary embodiments of the present invention that are not explicitly presented hereinafter can be specifically found in the corresponding sections.

### Example 1. Synthesis of Prodrug (Al-ProD)

### 1) Materials

Trt-Cl resin and all Fmoc amino acids were purchased from GL Biochem (Shanghai, China). Coupling reagents and cleavage cocktail reagents were purchased from Sigma Aldrich, and other solvents were purchased Daejung Chemical (Korea).

### 2) Synthesis of Prodrug (Al-ProD)

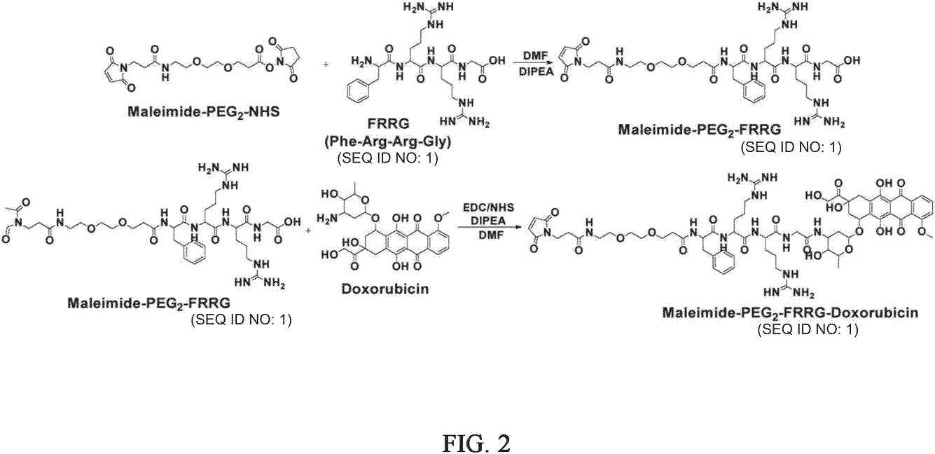

First, maleimide-PEG2-NHS (100 mg, 1 equiv) and NH2-FRRG(SEQ ID NO: 1)-COOH (251.4 mg, 2 equiv) were dissolved in anhydrous DMF (10 mL). Then, the solution was allowed to react at room temperature for 12 h to obtain maleimide-PEG2-FRRG(SEQ ID NO: 1)-COOH. The maleimide-PEG2-FRRG(SEQ ID NO: 1)-COOH (150 mg, 2 equiv), doxorubicin (48.2 mg, 1 equiv), EDC (44.1 mg, 4 equiv), and NHS (40.9 mg, 4 equiv) were dissolved in anhydrous DMF (10 mL). Thereafter, the solution was allowed to react at room temperature for 12 h. The reaction product, Al-ProD, was purified via HPLC (FIGS. 2 and 3).

### Example 2. Albumin-Prodrug Conjugate (Al-ProD+HSA)

A PBS solution (1 ml) containing 1% HSA was added to the prodrug (Al-ProD) synthesized in Example 1 and incubated at room temperature for 10 min to prepare an albumin-prodrug conjugate (Al-ProD+HSA).

### Example 3. Albumin-Prodrug Conjugate (Al-ProD+MSA)

An albumin-prodrug conjugate (Al-ProD+MSA) was prepared in the same manner as in Example 2, except that MSA was used instead of HSA.

### Example 4. Albumin-Prodrug Conjugate (Al-ProD+BSA)

An albumin-prodrug conjugate (Al-ProD+BSA) was prepared in the same manner as in Example 2, except that BSA was used instead of HSA.

### Experimental Example 1. Molecular Weight of the Prodrug (Al-ProD)

The molecular weight of the prodrug (Al-ProD) synthesized in Example 1 was analyzed by matrix-assisted laser desorption/ionization time-of-flight (MALDI-TOF) mass spectrometry (AB Sciex TOF/TOF 5800 System, USA) (with cyano-4-hydroycinnamic acid matrix).

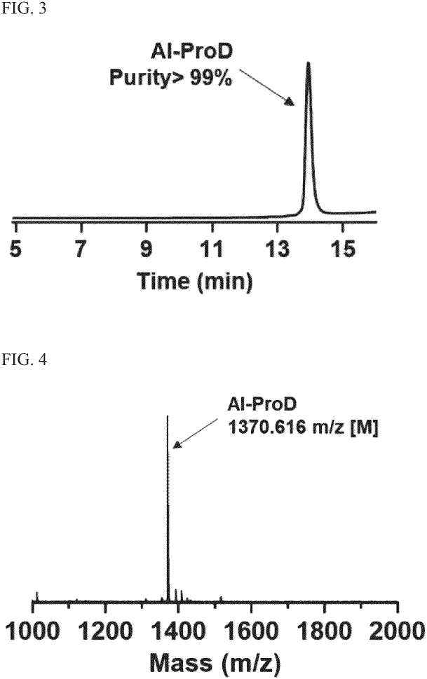

FIG. 4 shows the results of MALDI-TOF mass spectrometry for the prodrug (Al-ProD) synthesized in Example 1. Referring to FIG. 4, successful synthesis of the prodrug (Al-ProD) was confirmed. The molecular weight of the albumin-mediated prodrug (Al-ProD) was measured to be 1370.616 m/z [M].

### Experimental Example 2. Formation of Albumin-Prodrug (Al-ProD) Conjugates

It was analyzed whether the prodrug (Al-ProD) synthesized in Example 1 was covalently bound selectively and rapidly to albumin in situ to form an albumin-prodrug conjugate. To this end, a PBS solution (1 ml) containing 1% albumin (HSA, MSA or BSA) or 1% blocked albumin (blocked HSA, MSA or BSA) was added to 1 mg of the prodrug (Al-ProD) synthesized in Example 1 and incubated at room temperature for various times (0 h, 5 min, and 1 h). After completion of the incubation, samples were analyzed via SDS-PAGE and MALDI-TOF mass spectrometry.

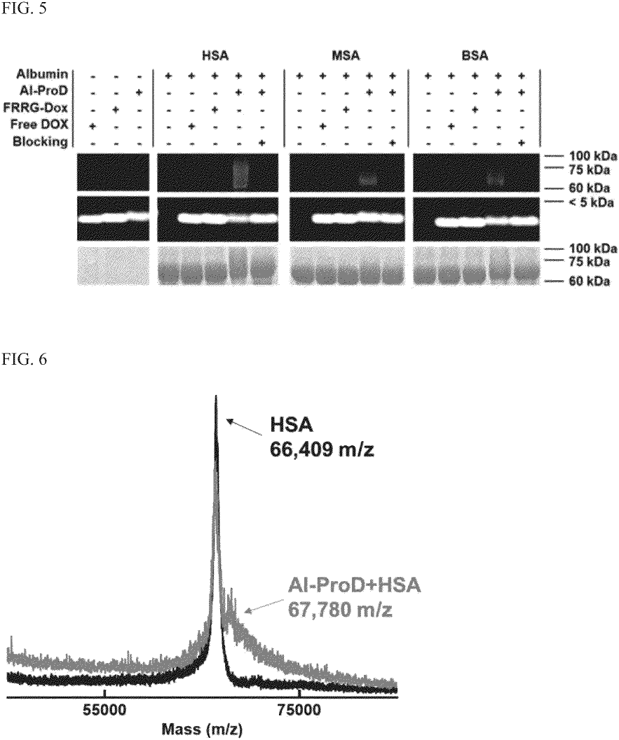

τ-maleimido butyric acid was added to 1% albumin (HSA, MSA or BSA) for 1 h to block the thiol group of the albumin. In the “Blocking” row in the table of FIG. 5, the plus signs (+) represent the use of blocked albumin.

For comparison, FRRG(SEQ ID NO: 1)-DOX without a maleimide group or doxorubicin (DOX) was used instead of the prodrug (Al-ProD). In the “FRRG(SEQ ID NO: 1)-DOX” and “Free DOX” rows in the table of FIG. 5, the plus signs (+) represent the use of FRRG(SEQ ID NO: 1)-DOX and DOX, respectively.

FIG. 5 shows the results of SDS-PAGE after incubation of the prodrug (Al-ProD) synthesized in Example 1 with albumin (HSA, MSA, BSA or blocked albumin) for 1 h.

As shown in FIG. 5, the prodrug (Al-ProD) synthesized in Example 1 was covalently bound rapidly to each albumin in situ to form an albumin-prodrug conjugate. The band of the prodrug (Al-ProD) was detected below 7 kDa but the band shifted to 50-75 kDa after incubation with albumin (HSA, MSA or BSA), indicating that the prodrug (Al-ProD) formed a conjugate with each albumin.

However, the band of the blocked albumin (HSA, MSA, BSA) was not detected at 50-75 kDa, indicating that the prodrug (Al-ProD) failed to form a conjugate with the blocked albumin. That is, the inventive prodrug (Al-ProD) selectively binds to albumin.

In contrast, FRRG(SEQ ID NO: 1)-DOX and DOX, whose structures are partially the same as that of the inventive prodrug (Al-ProD), did not form conjugates with any albumin.

FIG. 6 shows the results of MALDI-TOF analysis after incubation of the prodrug (Al-ProD) synthesized in Example 1 with albumin (HSA, MSA, BSA or blocked albumin) for 1 h.

As shown in FIG. 6, the peak of the HSA was shifted from 66,409 m/z to 67,780 m/z when incubated with the prodrug (Al-ProD). Considering that the peak of the prodrug (Al-ProD) was 1370.616 m/z, the prodrug (Al-ProD) synthesized in Example 1 was covalently bound rapidly and selectively to HSA in situ to form an albumin-prodrug conjugate.

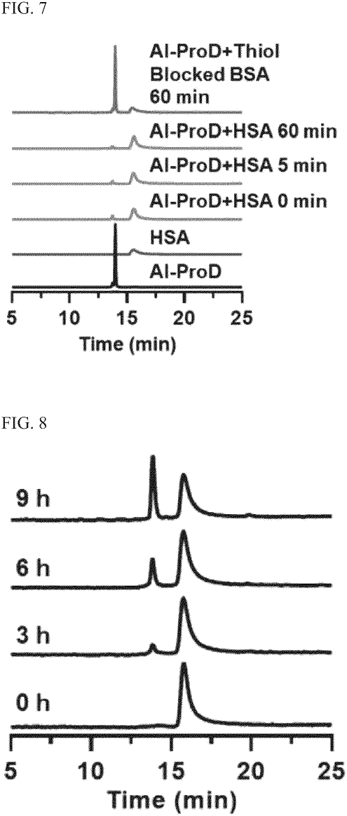

FIG. 7 shows the results of RP-HPLC analysis to determine whether an albumin-prodrug conjugate was formed by incubation of the prodrug (Al-ProD) synthesized in Example 1 with HSA or thiol-blocked HSA for various times (0 min, 5 min, and 60 min).

As shown in FIG. 7, the peaks of the prodrug (Al-ProD) and HSA were observed at 14 min and 16 min, respectively. When the prodrug (Al-ProD) was incubated with HSA, the peak of the prodrug (Al-ProD) shifted gradually to the peak of HSA in the HPLC spectrum, which is believed to be because the prodrug (Al-ProD) was bound to HAS to form an albumin-prodrug conjugate.

The conjugate formation began from the moment of mixing of the prodrug (Al-ProD) with HSA and was completed within a maximum of 5 min. When thiol-blocked HAS was used, an albumin-prodrug conjugate was not formed for 60 min. In conclusion, the prodrug (Al-ProD) does not form a bond with thiol-blocked albumin and selectively binds to the thiol group of albumin.

### Experimental Example 3. Selective Activation of the Prodrug (Al-ProD) By Cathepsin B

In this example, selective activation of the albumin-prodrug conjugate (Al-ProD+HSA) prepared in Example 2 by cathepsin B was investigated. Specifically, an investigation was made as to whether the albumin-prodrug conjugate (Al-ProD+HSA) of Example 2 was successfully cleaved by cathepsin B despite interference by albumin adjacent to the FRRG(SEQ ID NO: 1) peptide.

Enzyme reaction buffers containing different enzymes (cathepsin B, cathepsin E, cathepsin D, caspase-9, and caspase-3) (MES buffer; 50 µg/ml) were prepared. Each enzyme reaction buffer was added to the albumin-prodrug conjugate (Al-ProD+HSA) prepared in Example 2 and incubated at room temperature for various times (0 h, 3 h, 6 h, and 9 h). After completion of the incubation, samples were analyzed by HPLC. Physiological saline was used as a control instead of the enzyme. The control was marked with “Saline”.

FIG. 8 shows the results of HPLC analysis after the albumin-prodrug conjugate (Al-ProD+HSA) prepared in Example 2 was incubated with cathepsin B for various times (0 h, 3 h, 6 h, and 9 h).

As shown in FIG. 8, the albumin-prodrug conjugate (Al-ProD+HSA) prepared in Example 2 began to be cleaved to glycine-conjugated doxorubicin (G-DOX) from 3 h after treatment with cathepsin B and G-DOX release was gradually increased until 9 h.

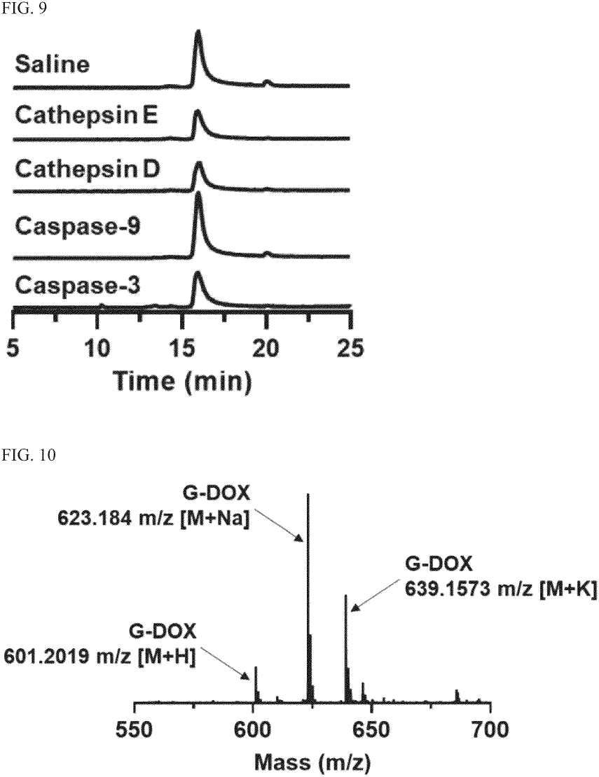

FIG. 9 shows the results of HPLC analysis after the albumin-prodrug conjugate (Al-ProD+HSA) prepared in Example 2 was incubated with various enzymes (cathepsin E, cathepsin D, caspase-9, and caspase-3) for various times (0 h, 3 h, 6 h, and 9 h);

As shown in FIG. 9, no significant numerical changes were observed for the albumin-prodrug conjugate (Al-ProD+HSA) prepared in Example 2 by enzymes (caspase-3, caspase-9, cathepsin D, and cathepsin E) other than cathepsin B, demonstrating that the albumin-prodrug conjugate (Al-ProD+HSA) did not release the inactivated form (i.e. the G-DOX molecule) in the presence of the enzymes other than cathepsin B.

In conclusion, the albumin-prodrug conjugate (Al-ProD+HSA) prepared in Example 2 is a highly stable and effective prodrug that is selectively activated only by cathepsin B.

FIG. 10 shows the results of MALDI-TOF mass spectrometry after the albumin-prodrug conjugate (Al-ProD+HSA) prepared in Example 2 was incubated with cathepsin B for 9 h. These results confirm the molecular weight of G-DOX (calculated mass: 600.58 Da, measured mass: 601.2019 m/z [M+H], 623.184 [M+Na], and 639.1573 [M+K]). It was clearly confirmed that the albumin-prodrug conjugate (Al-ProD+HSA) was effectively cleaved to albumin-FRR and G-DOX by cathepsin B. G-DOX is effectively metabolized into free-DOX by intracellular proteases to exhibit toxicity to cancer cells.

### Experimental Example 4. Determination of Cancer Cell-specific Anticancer Efficacy of the Albumin-Prodrug Conjugate (Al-ProD+HSA)

An in vitro cell experiment was conducted using breast cancer cells (MDA-MB-231) and rat cardiomyocytes (H9C2) to assess the cancer cell-specific activation and efficacy of the albumin-prodrug conjugate (Al-ProD+HSA).

2×10⁴ cancer cells (breast cancer cells (MDA-MB-231)) and normal cells (H9C2) were seeded in cell culture plates for confocal microscopy. As a control, MDA-MB-231 cells were treated and incubated with Cat-B-inhibitory siRNA for 24 h to inhibit the expression of cathepsin B (“MDA-MB-231 (+Cat-B inhibitor)”). After 24 h stabilization, cells were treated with the albumin-prodrug conjugate of Example 2 (Al-ProD+HSA) or free DOX at a concentration of 2 µM and cultured in an incubator at 37° C. for 48 h. Then, cells were washed with Dulbecco’s phosphate buffered saline (DPBS) to remove residual drug. Cells were treated with a cell fixative for 15 min and nuclei were stained blue by treatment with a DAPI solution for 10 min. The fluorescence of doxorubicin (DOX) was observed by confocal laser scanning microscopy (CLSM).

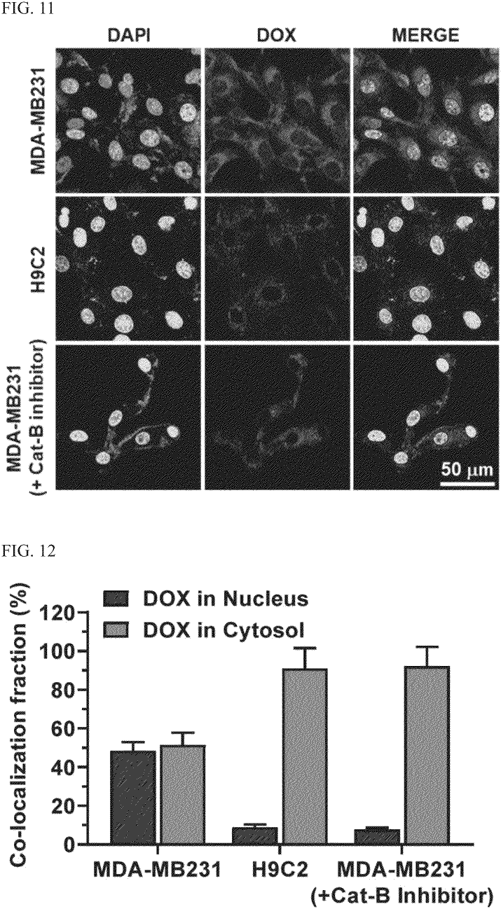

FIG. 11 shows confocal laser scanning microscopy (CLSM) images revealing the intracellular behaviors of doxorubicin (DOX) when MDA-MB-231, H9C2, and cathepsin B inhibitor-treated MDA-MB-231 were treated with the albumin-prodrug conjugate (Al-ProD+HSA) prepared in Example 2.

It is generally known that the expression level of cathepsin B is significantly higher in cancer cells than in normal cells. The expression level of cathepsin B in breast cancer cells (MDA-MB-231) was actually compared with that in rat cardiomyocytes (H9C2). As a result, MDA-MB-231 cells expressed a 24.26 ± 3.08-fold large amount of cathepsin B compared to H9C2 cells.

As shown in FIG. 11, both MDA-MB-231 and H9C2 cells showed intracellular uptake of the albumin-prodrug conjugate (Al-ProD+HSA). However, DOX fluorescence was observed only in the cytoplasm of H9C2 cells, but not in the nuclei of H9C2 cells. In contrast, large amounts of DOX fluorescence were observed in the nuclei as well as the cytoplasm of MDA-MB-231 cells. DOX fluorescence was observed only in the cytoplasm of cathepsin B inhibitor-treated MDA-MB-231 cells.

That is, since DOX is an anticancer drug that binds to DNA and induces toxicity, the results of intracellular behaviors reveal that the albumin-prodrug conjugate (Al-ProD+HSA) is specifically activated only in cancer cells overexpressing cathepsin B.

FIG. 12 shows the quantification analysis of DOX fluorescence in the nuclei or cytosol of MDA-MB-231, H9C2, and cathepsin B inhibitor-treated MDA-MB-231 after treatment with the albumin-prodrug conjugate (Al-ProD+HSA) prepared in Example 2.

As shown in FIG. 12, the albumin-prodrug conjugate (Al-ProD+HSA) showed 2.48-2.98-fold stronger fluorescence in the nuclei of cancer cells (MDA-MB-231) than the nuclei of other cells. That is, the albumin-prodrug conjugate (Al-ProD+HSA) did not enter the nuclei of normal cells and remained only in the cytoplasm but it effectively entered the nuclei of cancer cells.

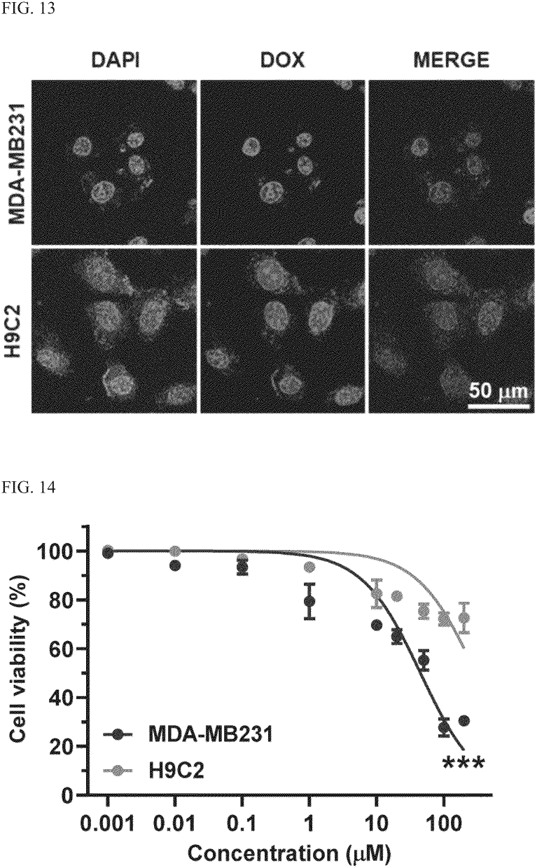

FIG. 13 shows confocal laser scanning microscopy (CLSM) images revealing the intracellular behaviors of doxorubicin (DOX) when MDA-MB-231, H9C2, and cathepsin B inhibitor-treated MDA-MB-231 were treated with free DOX.

As shown in FIG. 13, free DOX penetrated the nuclei of both cancer cells and normal cells, regardless of the type of cells. This indicates that there is no difference in the cellular behavior of free DOX in cancer cells and normal cells.

### Experimental Example 5. Cytotoxicity of the Albumin-Prodrug Conjugate (Al-ProD+HSA)

An in vitro cell experiment was conducted using breast cancer cells (MDA-MB-231) and rat cardiomyocytes (H9C2) to determine the cancer cell-specific cytotoxicity of the albumin-prodrug conjugate (Al-ProD+HSA). Specifically, cells were cultured and maintained in Dulbecco’s modified Eagle’s medium (DMEM) supplemented with 10% fetal bovine serum (FBS) and 1% penicillin/streptomycin in a humid atmosphere at 5% CO₂ and 95% air and at 37° C. 24 h after drug treatment, viable cells were counted using cell counting kit-8 (CCK-8) and used for further analysis.

Cancer cells (breast cancer cells (MDA-MB-231)) or normal cells (H9C2) were seeded at a density of 2×10⁴/well in 96-well cell culture plates. After 24 h stabilization, cells were treated with various concentrations (0.001, 0.01, 0.1, 1, 10, and 100 µM) of the albumin-prodrug conjugate (Al-ProD+HSA) of Example 2 or free DOX and cultured in an incubator at 37° C. for 48 h. Then, each well was treated with 10 µg of CCK solution, followed by incubation for 30 min. The absorbance of each 96-well plate was analyzed using a microplate reader (VERSAmax™, Molecular Devices Corp., Sunnyvale, CA) at 450 nm.

Student’s t-test was used for statistical analysis. Significance was indicated by *P < 0.05, **P < 0.01, ***P < 0.001.

FIG. 14 shows the cytotoxicities of the albumin-prodrug conjugate (Al-ProD+HSA) prepared in Example 2 to H9C2 and MDA-MB231 cells and FIG. 15 shows the cytotoxicities of free DOX to H9C2 and MDA-MB231 cells.

As shown in FIG. 14, the albumin-prodrug conjugate (Al-ProD+HSA) of Example 2 showed selective toxicity to cancer cells. Specifically, the IC₅₀ value of the albumin-prodrug conjugate (Al-ProD+HSA) of Example 2 in MDA-MB-231 cells was measured to be 7.33 µM, while it was > 200 µM in H9C2 cells. These results indicate that the albumin-prodrug conjugate (Al-ProD+HSA) is selective, specific, and deadly toxic to cancer cells but has significantly low toxicity to normal cells. In addition, the toxicity of the albumin-prodrug conjugate (Al-ProD+HSA) to normal cells was found to be different from that to cancer cells by a factor of at least 30.

As shown in FIG. 15, the conventional anticancer drug (free DOX) showed significant cytotoxicities in both MDA-MB231 cells and H9C2 cells when used alone.

Taken the results together, the albumin-prodrug conjugate (Al-ProD+HSA) of Example 2 is selective to cancer cells and exhibits specific anticancer effects. In addition, the albumin-prodrug conjugate (Al-ProD+HSA) of Example 2 showed only minimal toxicity to normal cells even at high concentrations (≥ 10 µM), indicating that the albumin-prodrug conjugate exerts superior anticancer effects with minimal side effects.

### Experimental Example 6. Analysis of Pharmacokinetics of the Prodrug (Al-ProD)

One of the major failure factors in drug development is attributed to unsuitable pharmacokinetics. Here, an experiment was conducted to investigate whether the inventive prodrug (Al-ProD) exhibits suitable pharmacokinetics in vivo.

When administered, the inventive prodrug (Al-ProD) binds to albumin present in blood vessels in vivo to form an albumin-prodrug conjugate (Al-ProD+HSA) and specifically accumulates in cancer tissues. Thus, the inventive prodrug (Al-ProD) was intravenously administered to BALB/c nude mice and its pharmacokinetics were analyzed.

All animal experiments were conducted in accordance with the guidelines approved by the Institutional Animal Care and Use Committee of Korea Institute of Science and Technology (KIST). BALB/c nude mice (5.5 weeks old, 20-25 g, male) purchased from Nara Bio INC (Gyeonggi-do, Korea) were used as animal models. 1×10⁷ MDA-MB-231 cells were inoculated into the left thigh of each of the mice (n=6) to construct establish a cancer animal model. 5 weeks after inoculation, experiments were conducted when cancer volumes were 250-300 mm³.

When cancer volumes in the cancer animal models reached 250-300 mm³, the prodrug (Al-ProD) of Example 1 (an equimolar amount to 3 mg/kg doxorubicin) or free DOX (3 mg/kg) was injected into each animal model via the tail vein. After administration of the drugs, noninvasive near-infrared fluorescence (NIRF) imaging data were obtained using an in vivo fluorescence imaging system (IVIS Luminar III) to assess the in vivo tumor accumulation of the prodrug (Al-ProD). The areas under the curves of the time-concentration curves (AUCs) corresponding to the near-infrared data were calculated using Origin 2020 software.

Animal models were sacrificed 12 h after injection of the drugs and organs (liver, lung, spleen, kidney, and heart) and cancer tissues were excised therefrom. The amounts of the drugs remaining in the major organs and cancer tissues were calculated via fluorescence imaging using IVIS Luminar III. The fluorescence intensities were quantified using the Living Image software (PerkinElmer, USA) and plotted.

The organs and cancer tissues excised from the animal models were sliced into 10 µm thick sections and treated with DAPI solution for 10 min to stain the cell nuclei blue. The fluorescence of DOX or DAPI from the sections was observed using a confocal laser scanning microscope (CLSM).

Two groups of animal models
Group 1 (Free DOX): Cancer animal models administered free DOX (3 mg/kg) via the tail vein
Group 2 (Al-ProD): Cancer animal models administered the prodrug of Example 1 (Al-ProD) (an equimolar amount to 3 mg/kg doxorubicin) via the tail vein
Student’s t-test was used for statistical analysis. Significance was indicated by *P < 0.05, **P < 0.01, ***P < 0.001.

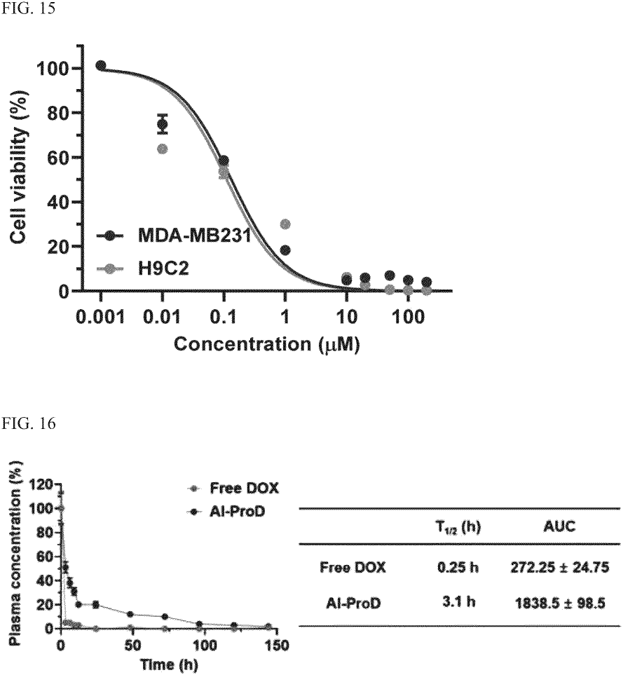

FIG. 16 shows the pharmacokinetics (PK) of the prodrug (Al-ProD) synthesized in Example 1 (Group 2) or free DOX (3 mg/kg, Group 1) after intravenous administration to cancer animal models.

As shown in FIG. 16, free DOX rapidly disappeared in vivo within 15 min. In contrast, the prodrug (Al-ProD) of Example 1 remained in vivo for 50-150 h and showed an extended half-life (T_{½} (h)) of ≥ 3 h.

The in vivo residence time of the prodrug (Al-ProD) of Example 1 was dramatically extended by at least 12 times compared to that of the simple anticancer drug.

As can be seen from the results in the table on the right side of FIG. 16, the area under the curve (AUC) of the prodrug (Al-ProD) of Example 1 was 7-fold increased compared to that of free DOX.

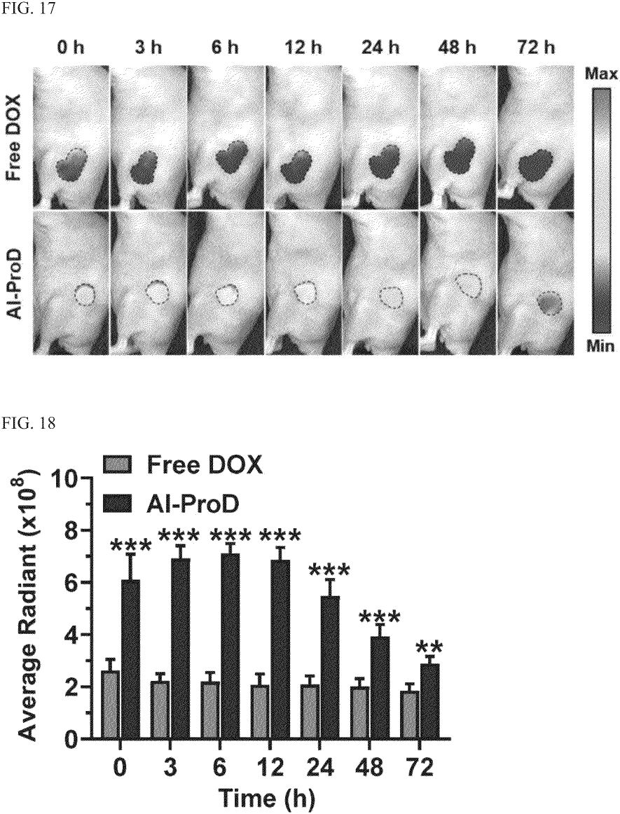

FIG. 17 shows noninvasive near-infrared fluorescence (NIRF) images of the groups intravenously administered the prodrug (Al-ProD) synthesized in Example 1 or free DOX (3 mg/kg) and FIG. 18 shows the quantification analysis of fluorescence from tumor tissues in the NIRF images.

As shown in FIG. 17, a small quantity of free DOX was accumulated in cancer tissues due to its short in vivo half-life and even it was decreased rapidly in a short time. In contrast, the fluorescence from cancer tissues was 3.33-4.08-fold stronger for 0-72 h in the group administered the prodrug (Al-ProD) of Example 1 than in the group administered free DOX and a considerable quantity of DOX was retained in cancer tissues even after 72 h. These results are believed to be due to the structural features of the prodrug (Al-ProD) of Example 1 that rapidly binds to albumin in the blood to form a conjugate when administered in vivo to achieve albumin-mediated cancer cell targeting.

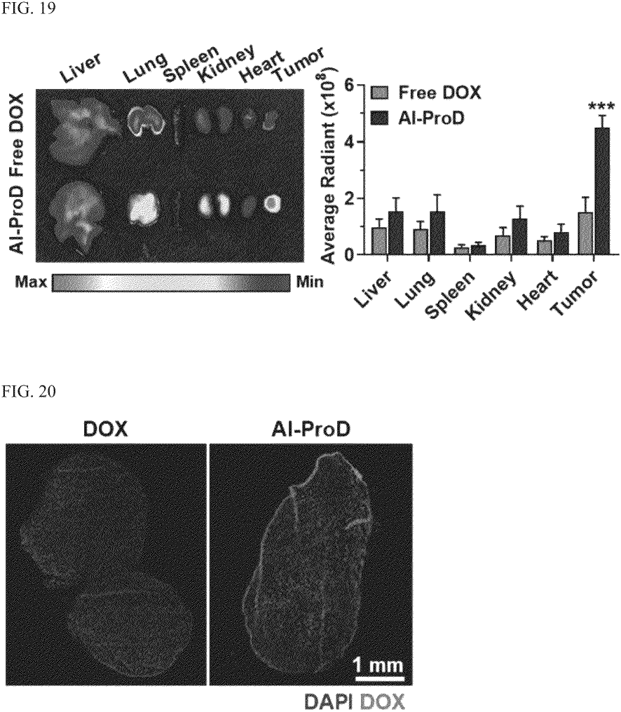

FIG. 19 shows (right) noninvasive near-infrared fluorescence (NIRF) images of major organs (liver, lung, spleen, kidney, and heart) and cancer tissues excised from the groups 12 h after intravenous administration of the prodrug (Al-ProD) synthesized in Example 1 or free DOX (3 mg/kg) and (left) quantification analysis of fluorescence from the cancer tissues in the NIRF images.

As shown in FIG. 19, Group 2 administered the prodrug (Al-ProD) of Example 1 showed 3.41-4.92-fold stronger DOX fluorescence from cancer tissues than Group 1 administered free DOX (3 mg/kg). These results indicate that the prodrug (Al-ProD) of Example 1 has an outstanding ability to selectively accumulate in cancer tissues, unlike the single anticancer drug (free DOX).

FIG. 20 shows confocal laser scanning microscopy (CLSM) images of DAPI-stained cancer tissues excised from the groups intravenously administered the prodrug (Al-ProD) synthesized in Example 1 or free DOX (3 mg/kg).

As shown in FIG. 20, the group administered the prodrug (Al-ProD) of Example 1 showed strong DOX fluorescence from cancer tissues due to the ability of the prodrug to accumulate in cancer tissues, while a very small quantity of free DOX (3 mg/kg) was present in cancer tissues.

The above experimental results concluded that binding of the prodrug (Al-ProD) of Example 1 to albumin in vivo significantly extends the in vivo half-life of the prodrug by at least 3 times and can induce high accumulation of the prodrug in cancer tissues.

### Experimental Example 7. Evaluation of Anticancer Efficacy and Toxicity Of the Prodrug (Al-ProD)

The inventive prodrug (Al-ProD) binds to endogenous albumin present in blood vessels in vivo to form an albumin-prodrug conjugate (Al-ProD+HSA), ensuring an outstanding ability of the anticancer prodrug to target cancer cells and high stability of the anticancer prodrug in normal cells. An in vivo animal experiment was conducted using BALB/c nude mice to determine the anticancer efficacy of the inventive prodrug (Al-ProD) and the toxicity (side effect) of the inventive prodrug (Al-ProD) to normal cells.

Specifically, all animal experiments were conducted in accordance with the guidelines approved by the Institutional Animal Care and Use Committee of Korea Institute of Science and Technology (KIST). BALB/c nude mice (5.5 weeks old, 20-25 g, male) purchased from Nara Bio INC (Gyeonggi-do, Korea) were used as animal models. 1×10⁷ MDA-MB-231 cells were inoculated into the left thigh of each of the mice (n=6) to construct establish a cancer animal model. 5 weeks after inoculation, experiments were conducted when cancer volumes were 250-300 mm³.

When cancer volumes in the cancer animal models reached 250-300 mm³, the prodrug (Al-ProD) of Example 1 (an equimolar amount to 3 mg/kg doxorubicin) (Group 2), free DOX (3 mg/kg, Group 1) or physiological saline (3 mg/kg, Group 3) was injected into each animal model via the tail vein. Changes in body weight and cancer tissue volume of each group were measured and survivals were analyzed every two days from immediately after injection (day 0). The cancer tissue volume (V; mm³) was calculated as 0.53 × largest diameter × (smallest diameter)². After 20 days, each group was euthanized. Major organs (liver, lung, spleen, kidney, and heart) and cancer tissues were excised and sliced into 10 µm thick sections for histological assays.

Specifically, the tissues were fixed with 10% formalin, embedded in paraffin, and sectioned to a thickness of 10 µm. The tissue sections were stained with H&E, terminal deoxynucleotidyl transferase dUTP nick-end labeling (TUNEL), and 4,6-diamidino-2-phenylindole (DAPI, Sigma-Aldrich). Structural changes and toxicities in the tissue sections were evaluated using a ZEISS light microscope or fluorescence microscope.

Three groups of animal models
Group 1 (Free DOX): Cancer animal models administered free DOX (3 mg/kg) via the tail vein
Group 2 (Al-ProD): Cancer animal models administered the prodrug of Example 1 (Al-ProD) (molar dose equivalent to 3 mg/kg doxorubicin) via the tail vein
Group 3 (Saline): Cancer animal models administered 3 mg/kg physiological saline through the tail vein
Student’s t-test was used for statistical analysis. Significance was indicated by *P < 0.05, **P < 0.01, ***P < 0.001.

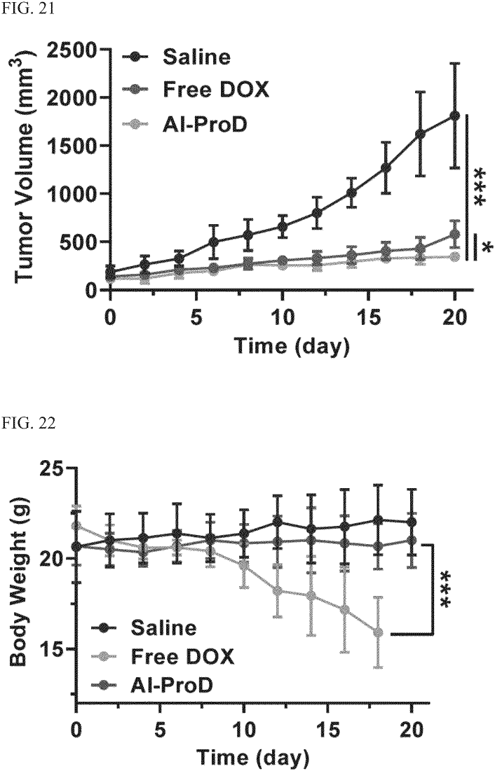

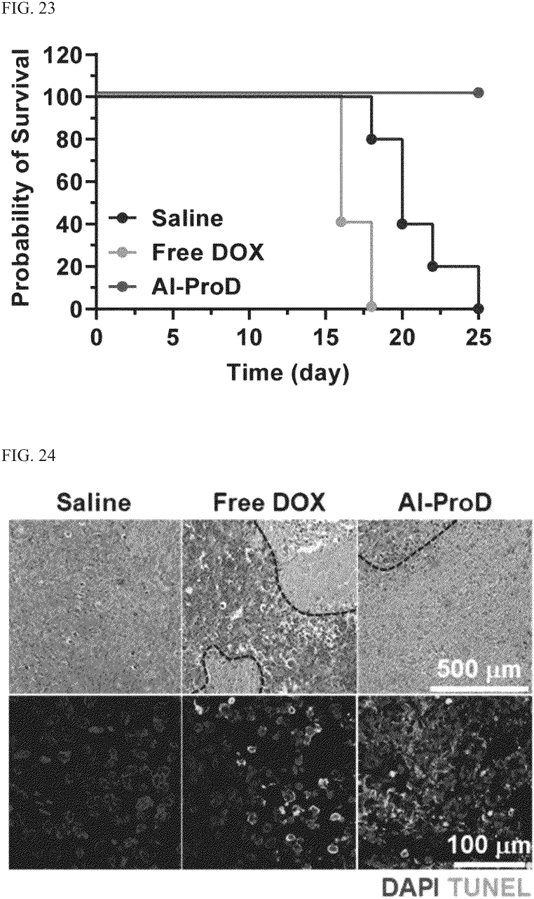

FIG. 21 shows time-dependent changes in the cancer volume (V; mm³) of the animal cancer models after treatment with the prodrug (Al-ProD) synthesized in Example 1, free DOX or physiological saline. FIG. 22 shows time-dependent changes in the body weight of the cancer animal models after treatment with the prodrug (Al-ProD) synthesized in Example 1, free DOX or physiological saline. FIG. 23 shows time-dependent changes in the survival of the cancer animal models after treatment with the prodrug (Al-ProD) synthesized in Example 1, free DOX or physiological saline.

As shown in FIG. 21, the cancer volumes of the cancer animal models in Group 2 treated with the prodrug (Al-ProD) of Example 1 were 347.42 ± 25.9 mm³, and the cancer volumes of the cancer animal models in Groups 1 and 3 treated with free DOX and saline were 580.25 ± 139.92 mm³ (P < 0.05) and 1810.98 ± 544.56 mm³ (P < 0.001), respectively. These results demonstrate that the prodrug (Al-ProD) of Example 1 significantly inhibites cancer growth compared to free DOX.

As shown in FIG. 22, no significant changes in the body weights of the cancer animal models in Group 2 treated with the prodrug (Al-ProD) of Example 1 were observed, but the cancer animal models in Group 1 treated with free DOX showed significant body weight losses. That is, free DOX caused severe systemic toxicity and even death within 18 days, whereas the prodrug (Al-ProD) of Example 1 could achieve high anticancer efficacy without causing systemic toxicity.

As shown in FIG. 23, the cancer animal models in Group 2 treated with the prodrug (Al-ProD) of Example 1 survived over 25 days and none of them died. In contrast, more than half of the cancer animal models in Group 1 treated with free DOX died from systemic toxicity caused by the drug rather than cancer on day 15 and the cancer animal models were all dead within 18 days. The cancer animal models in Group 3 treated with physiological saline (control group; Saline) began to die from day 20 and all died of cancer within 25 days.

In summary, the inventive prodrug (Al-ProD) forms a conjugate by in vivo injection without a separate process for forming a conjugate with albumin. In addition, due to its structural features, the inventive prodrug (Al-ProD) effectively inhibits only the growth of cancer cells without causing any side effects in normal cells to offer chemotherapeutically stable prophylactic or therapeutic efficacy against cancer.

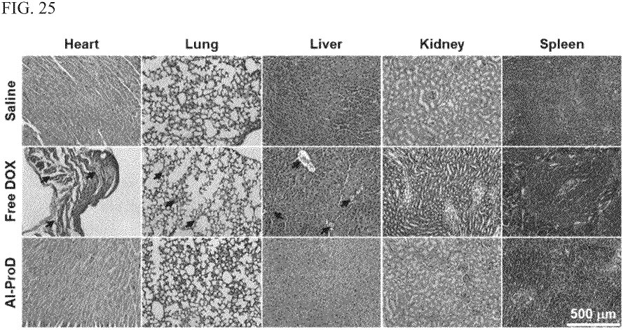

FIG. 24 shows microscopy images of H&E- and TUNEL-stained sections of cancer tissues excised from the cancer animal models sacrificed on day 20 after treatment with the prodrug synthesized in Example 1 (Al-ProD), free DOX or physiological saline; As shown in FIG. 24, the treatment with the prodrug (Al-ProD) of Example 1 in Group 2 induced significant damage to and apoptosis of cancer tissues compared to the treatment with free DOX in Group 1 and the treatment with saline in Group 3. That is, the binding of the prodrug (Al-ProD) of Example 1 to albumin in vivo made the prodrug (Al-ProD) stable in normal cells and allowed the prodrug (Al-ProD) to exert potent anticancer efficacy against cancer cells.

FIG. 25 shows images of H&E-stained sections of major tissues excised from the cancer animal models sacrificed on day 20 after treatment with the prodrug of Example 1 (Al-ProD), free DOX or physiological saline.

As shown in FIG. 25, no significant damage to the H&E-stained normal tissues (heart, lung, liver, kidney, and spleen) was observed in Group 2 treated with the prodrug of Example 1 (Al-ProD), whereas significant structural abnormalities were observed in all tissues of the cancer animal models in Group 1 treated with free DOX.

What is claimed is:

1. An anticancer prodrug comprising a peptide having the amino acid sequence set forth in SEQ ID NO: 1, a maleimide group conjugated to the N-terminus of the peptide, and an anticancer drug conjugated to the C-terminus of the peptide.

2. The anticancer prodrug according to claim 1, further comprising a linker between the N-terminus of the peptide and the maleimide group.

3. The anticancer prodrug according to claim 2, wherein the linker is represented by *—(CH₂)₂—C(═O)—NH—(CH₂CH₂O)ₙ—CH₂CH₂—C(═O)—* wherein n is an integer from 1 to 10 and each asterisk * represents a binding site.

4. The anticancer prodrug according to claim 1, wherein the anticancer prodrug has a molecular weight of 1 to 5 kDa.

5. The anticancer prodrug according to claim 1, wherein the anticancer prodrug covalently binds selectively and rapidly to albumin present in the blood in situ to form an albumin-prodrug conjugate when administered intravenously.

6. The anticancer prodrug according to claim 5, wherein the albumin-prodrug conjugate may have a molecular weight of 65 to 70 kDa.

7. The anticancer prodrug according to claim 5, wherein the albumin-prodrug conjugate has an in vivo half-life of 2 to 5 hours.

8. The anticancer prodrug according to claim 1, wherein the anticancer prodrug is activated by cathepsin B overexpressed in cancer cells.

9. The anticancer prodrug according to claim 1, wherein the anticancer drug is selected from the group consisting of doxorubicin, cyclophosphamide, mechlorethamine, uramustine, melphalan, chlorambucil, ifosfamide, bendamustine, carmustine, lomustine, streptozocin, busulfan, dacarbazine, temozolomide, thiotepa, altretamine, duocarmycin, cisplatin, carboplatin, nedaplatin, oxaliplatin, satraplatin, triplatin tetranitrate, 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thioguanine, camptothecin, topotecan, irinotecan, etoposide, teniposide, mitoxantrone, paclitaxel, docetaxel, ixabepilone, vinblastine, vincristine, vindesine, vinorelbine, estramustine, maytansine, mertansine (DM1), DM4, dolastatin, auristatin E, auristatin F, monomethyl auristatin E, monomethyl auristatin F, derivatives thereof, and combinations thereof.

10. The anticancer prodrug according to claim 1, wherein the anticancer prodrug is represented by Formula 1: 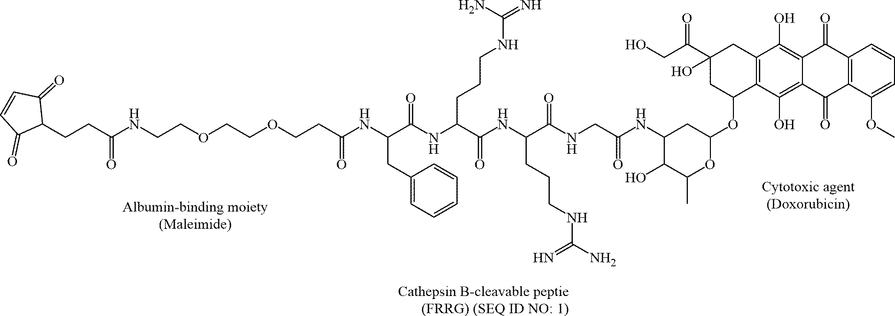
.

11. A pharmaceutical composition comprising the anticancer prodrug according to claim 1 as an active ingredient.

12. The pharmaceutical composition according to claim 11, wherein the pharmaceutical composition is for use in preventing or treating cancer.

13. The pharmaceutical composition according to claim 12, wherein the pharmaceutical composition is administered by intravenous injection.

14. The pharmaceutical composition according to claim 12, wherein the cancer is selected from the group consisting of gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, cervical cancer, bone cancer, non-small cell bone cancer, hematologic malignancy, skin cancer, head or neck cancer, uterine cancer, rectal cancer, perianal cancer, fallopian tube cancer, endometrial cancer, vaginal cancer, vulvar cancer, Hodgkin’s disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater cancer, peritoneal cancer, tongue cancer, small cell cancer, pediatric lymphoma, neuroblastoma, duodenal cancer, ureteral cancer, astrocytoma, meningioma, renal pelvis cancer, vulvar cancer, thymus cancer, central nervous system (CNS) tumor, primary central nervous system lymphoma, spinal cord tumor, brainstem glioma, pituitary adenoma, and combinations thereof.

15. A method for preventing or treating cancer in a subject in need thereof, comprising a step of administering the pharmaceutical composition according to claim 11.

16. The method according to claim 15, wherein the administering is intravenous injection.

17. The method according to claim 15, wherein the cancer is selected from the group consisting of gastric cancer, lung cancer, non-small cell lung cancer, breast cancer, ovarian cancer, liver cancer, bronchial cancer, nasopharyngeal cancer, laryngeal cancer, pancreatic cancer, bladder cancer, colon cancer, cervical cancer, bone cancer, non-small cell bone cancer, hematologic malignancy, skin cancer, head or neck cancer, uterine cancer, rectal cancer, perianal cancer, fallopian tube cancer, endometrial cancer, vaginal cancer, vulvar cancer, Hodgkin’s disease, esophageal cancer, small intestine cancer, endocrine gland cancer, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, salivary gland cancer, sarcoma, pseudomyxoma, hepatoblastoma, testicular cancer, glioblastoma, lip cancer, ovarian germ cell tumor, basal cell carcinoma, multiple myeloma, gallbladder cancer, choroidal melanoma, ampulla of Vater cancer, peritoneal cancer, tongue cancer, small cell cancer, pediatric lymphoma, neuroblastoma, duodenal cancer, ureteral cancer, astrocytoma, meningioma, renal pelvis cancer, vulvar cancer, thymus cancer, central nervous system (CNS) tumor, primary central nervous system lymphoma, spinal cord tumor, brainstem glioma, pituitary adenoma, and combinations thereof.


SEQUENCE LISTING (1 sequence)

SEQ ID NO: 1 - protein, 4 aa, Artificial Sequence
  cathepsin B cleavable peptide
FRRG